(19) United States
(12) Patent Application Publication
(10) Pub. No.: US 2024/0148527 A1
(43) Pub. Date: May 9, 2024

(54) SYSTEMS AND METHODS FOR TREATMENT OF AIRWAY BLOCKAGES

(71) Applicant: Merit Medical Systems, Inc., South Jordan, UT (US)

(72) Inventors: Zeke Eller, Plano, TX (US); Barton Gill, South Jordan, UT (US); Nick Poulides, South Jordan, UT (US)

(21) Appl. No.: 18/500,004

(22) Filed: Nov. 1, 2023

Related U.S. Application Data
(60) Provisional application No. 63/382,218, filed on Nov. 3, 2022.
(60) Provisional application No. 63/481,743, filed on Jan. 26, 2023.

(51) Int. Cl. A61F 2/95

(52) U.S. Cl. CPC A61F 2/9517

(57) ABSTRACT

A method for treating a patient's airway by coupling a deployment device to a bronchoscope at the working channel is disclosed. The coupling of such can hold the deployment device in a fixed position and allow for the precise placement of small airway stents within the lungs of a patient by a single practitioner. The fixed position may be adjustable over a range.

### CROSS-REFERENCE TO RELATED APPLICATIONS

This application claims priority to U.S. Provisional Patent Application No. 63/382,218 filed on Nov. 3, 2022 and U.S. Provisional Patent Application No. 63/481,743 filed on Jan. 26, 2023, the disclosures of which are incorporated herein, in their entireties, by this reference.

### TECHNOLOGICAL FIELD

The present disclosure generally relates medical devices including stents and stent delivery systems. In some embodiments, this disclosure relates to the treatment of airway blockages using an airway stent. Further, in some embodiments, it is related to the use of covered airway stents in a precise insertion method.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates of a bronchoscope having an adapter, according to an embodiment.

FIG. 1B illustrates a portion of the bronchoscope of FIG. 1A with the adapter removed to expose a first fitting, according an embodiment.

FIG. 1C illustrates a second fitting replacing the first fitting on the bronchoscope of FIG. 1A, according to an embodiment.

FIG. 1D illustrates an embodiment of a deployment device secured to the second fitting on the bronchoscope of FIG. 1C.

FIG. 1E illustrates a cross-sectional view of an embodiment of a third fitting configured to be secured to the bronchoscope of FIG. 1A and a deployment device secured to the third fitting, according to an embodiment.

FIG. 2A illustrates an embodiment of a deployment device and an airway stent.

FIG. 2B illustrates an alternative embodiment of a deployment device and an airway stent.

FIG. 3 illustrates an embodiment of an airway stent.

FIG. 4 illustrates a position of a deployed airway stent in accordance with certain embodiments.

FIG. 5 illustrates a deployed airway stent at an airway junction in accordance with certain embodiments.

FIG. 6 illustrates a process flow of inserting and deploying an airway stent.

FIG. 7 illustrates an alternative process flow of the deployment of an airway stent in accordance with certain embodiments.

FIG. 8 illustrates a process flow of the deployment of an airway stent in accordance with certain embodiments.

### DETAILED DESCRIPTION

Airway stents may be deployed to treat some patients that may require the use of a mechanical device to help open a portion of the bronchial tubes or other passages within the lungs. The lungs are full of airway passages that allow for the exchange of oxygen and carbon dioxide with the blood of a person as they breathe. The airway passages are referred to as bronchioles and are like branches of a tree that branch off from larger sections into smaller and smaller sections until the oxygen taken in reaches a small end of the branch called the alveoli, where gas exchange takes place in the lungs.

Patients can sometimes experience problems that can cause blockages within any portion of the bronchial tubes, bronchiole, or other passages that can hinder a person's ability to adequately supply oxygen to their blood. This can lead to a whole host of medical problems. Accordingly, it is highly desirable to allow for free flow of oxygen into the lungs.

As used herein, the term airway refers to any passageway or lumen in the respiratory system, including bronchial tubes, bronchiole, and so forth. Additionally, though specific examples recited herein may refer to a particular airway or portion of the respiratory system, the concepts are also applicable to any portion of the respiratory system as well as other areas of the body, as further detailed below.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

While certain examples in the present disclosure recite airway stents or delivery of a stent via passages of the respiratory system, other medical appliances delivered in other areas of the body are likewise within the scope of this disclosure. For example, esophageal stents, pyloric stents, colonic stents, biliary stents, transluminal stents, vascular stents, as well as other types of medical appliances are within the scope of this disclosure. Furthermore, deployment devices (e.g., delivery devices) and methods designed for a high level of physician control and accurate deployment, such as the deployment devices (e.g., delivery devices) and methods disclosed herein, may be used with airway stents, esophageal stents, pyloric stents, colonic stents, biliary stents, transluminal stents, vascular stents, and other medical devices are all within the scope of this disclosure. Further, steps and devices for coupling a deployment device (e.g., stent delivery device or system) to a bronchoscope may also be applied to coupling a delivery system to another introducer system, such as a vascular sheath in the case of vascular stents.

Turning now to the figures, many embodiments can be directed towards a method of treating an airway obstruction or problem through the insertion and deployment of an airway stent. Many embodiments can include utilizing a bronchoscope and connecting or attaching an airway stent deployment device to the bronchoscope. Use of reusable bronchoscopes as well as disposable bronchoscopes are within the scope of this disclosure. The airway stent deployment device can be attached by a variety of mechanisms, however, many embodiments can include the use of an adapter that can allow for the deployment device to be attached and supported by the body of the bronchoscope, including embodiments wherein the deployment device is coupled to the biopsy port, working channel, or other portions of the bronchoscope. This configuration can allow for the physician to control both the bronchoscope and the deployment device with minimal movement to the deployment device without assistance from a second practitioner such as a nurse or technician. Accordingly, the airway stent can be precisely inserted into the patient's airway at the desired location without undesired movement, repositioning, or assistance from a second practitioner. Some embodiments can also include the use of covered stents and/or stents that have apertures or openings to allow for gas exchange to occur through the stent.

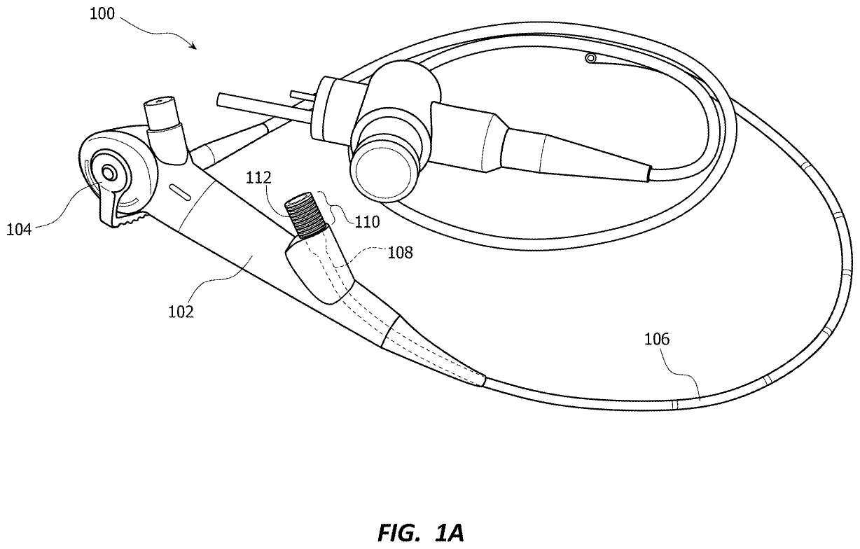

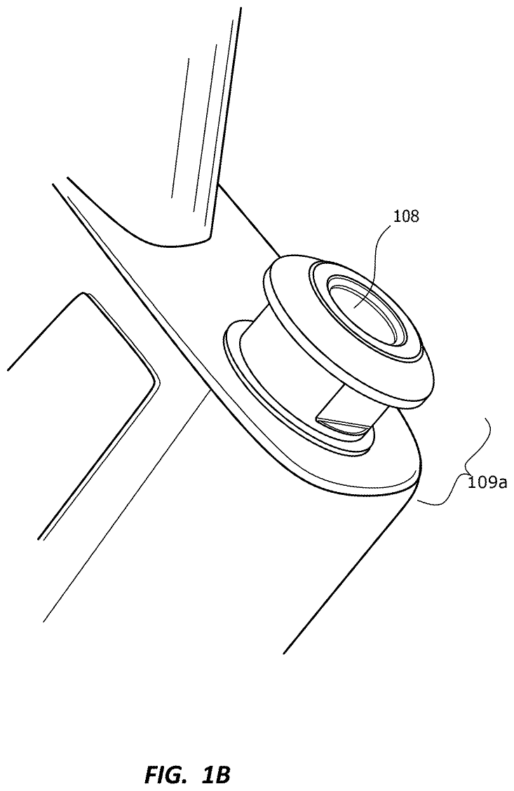

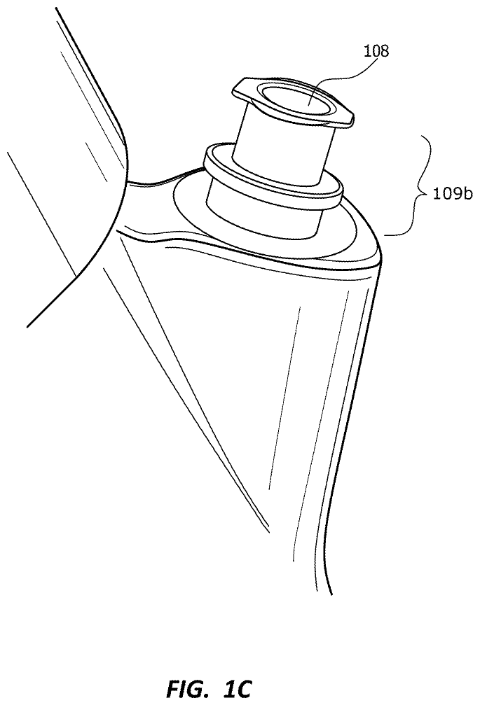

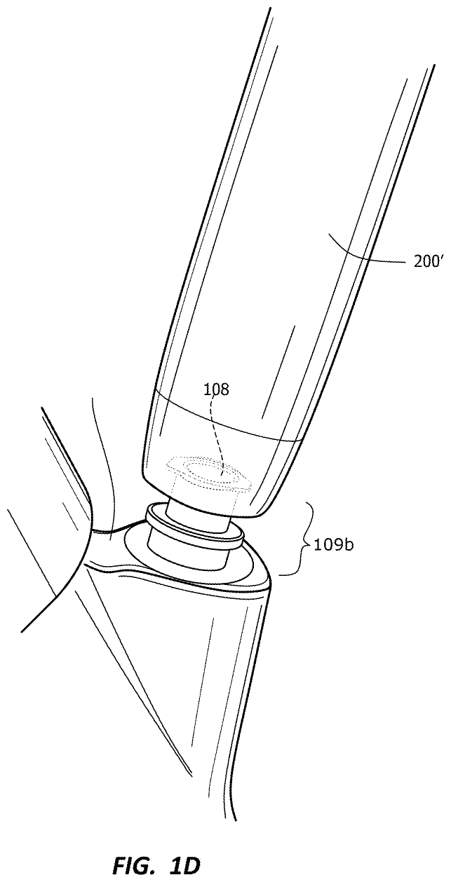

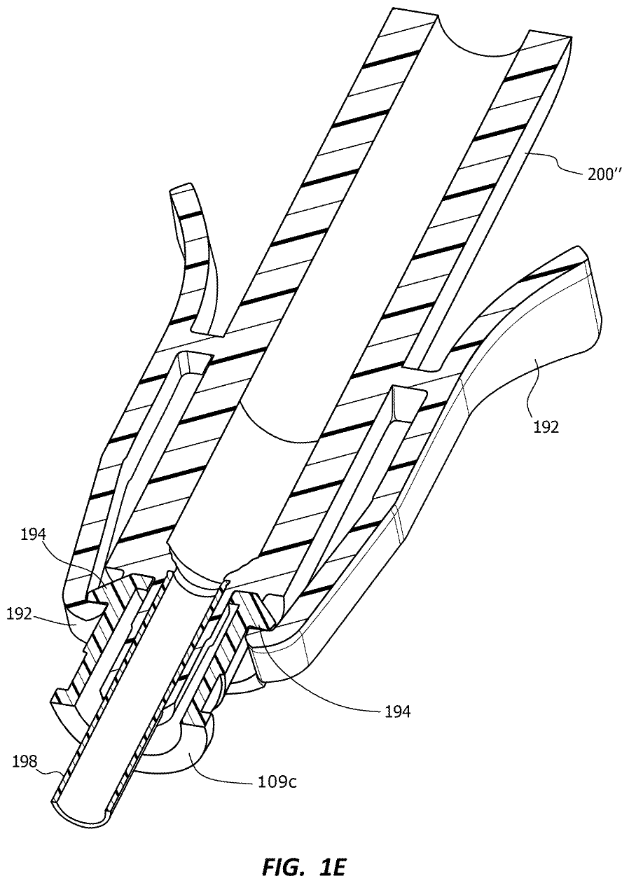

Airway stents have been used in a variety of settings to treat a number of different illnesses. Airway stents can be made from a variety of materials including metal and/or silicone. In some therapies an airway stent is inserted into the airway using a bronchoscope, such as the bronchoscope 100 illustrated in FIG. 1A. The bronchoscope 100 can have a main body portion 102 that has a control unit 104 at one end and an insertion cord 106 at the other end. The tube or insertion cord 106 of the bronchoscope can be inserted into a person's airway and can have a number of different components such as a camera, light source, working channel, suction channel, and/or a grasping tool.

The main body 102 can also have a working channel 108 in fluid communication with the insertion cord 106. The working channel 108 may extend from the insertion cord 106 at least partially towards the control unit 104. In many embodiments, the main body portion 102 includes a protrusion that extends off of the main body. The working channel 108 may extend through the protrusion and provide an opening by which a doctor can insert an introducing device to deploy elongate instruments, including deployment devices (e.g., delivery devices) for an airway stent. In some configurations, the control unit portion 104 can have an eye piece or be connected to an external display system to allow the doctor to see and control the movement of the insertion cord in the patient.

Although the typical bronchoscope can allow for the use of secondary devices (e.g., such as airway stents delivery devices), bronchoscopes are not equipped with an interface that can allow the secondary devices to be fixed to the bronchoscope while in use. A deployment device (e.g., a stent delivery device or other delivery device) or other treatment device may comprise an elongated member that may be required to bend and/or shape around one or more corners when being inserted. As such the bending can cause tension, compression, friction, or other forces on the deployment device which can interfere with the operation or placement of the deployment device. For example, friction can prevent the outer sheath of the deployment device from retracting or from retracting in a smooth and controlled manner, which can in turn result in unpredictable distal advancement of the inner catheter and stent, and/or imprecise deployment of the stent. Thus, this friction can result in inaccurate stent deployment. Inaccurate stent placement may extend procedure times, cause patient injury, and require stent repositioning or remove. In the case of airway stents, if the stent is misplaced, the misplaced stent can cause a blockage or partial blockage of an adjacent airway in the lungs, which can result in more problems.

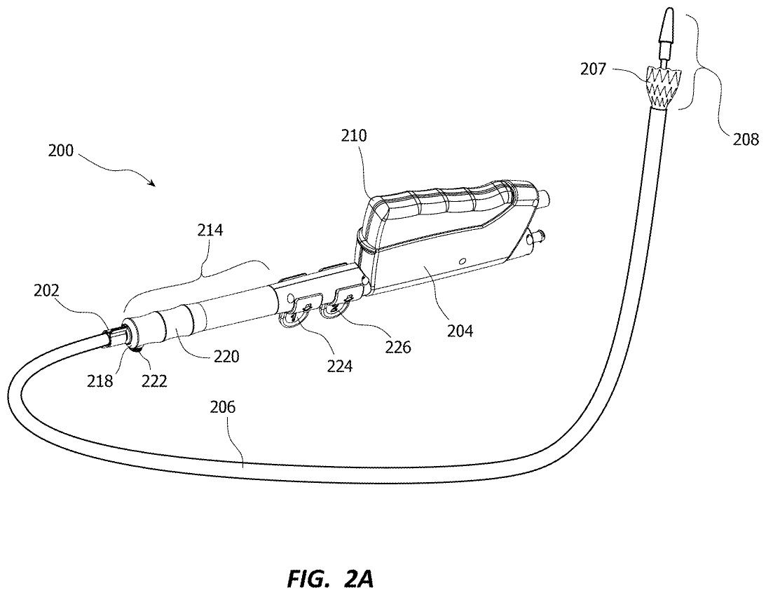

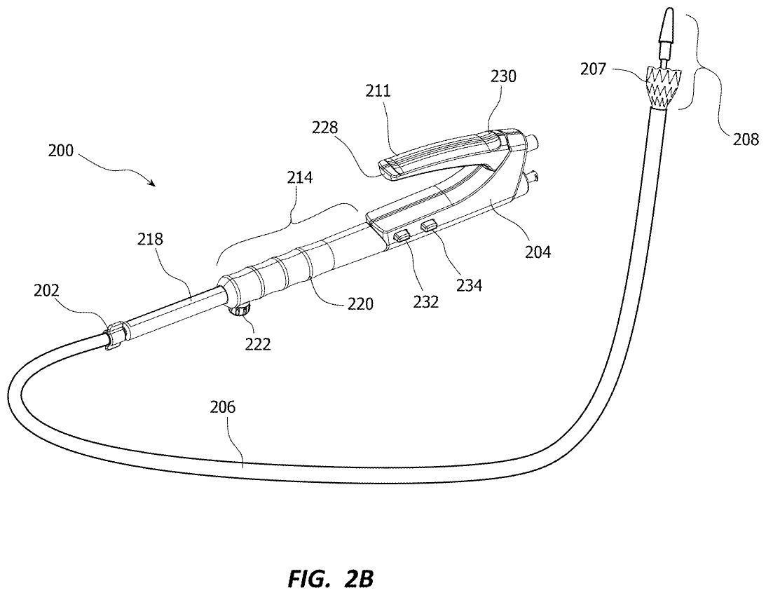

In some instances, an adapter component can be used to couple a deployment device (e.g., delivery device) to a bronchoscope. In some embodiments such adapters may comprise a separate component that attaches to an open end of the working channel and provides an attachment end that can be engaged with the introducing or deployment device. The coupler or adapter can have a lumen that can allow for a portion of the introducing or deployment device to be inserted and guided into the bronchoscope. FIGS. 2A and 2B, for example, illustrate exemplary embodiments of an introducing or deployment device 200 that can be coupled to a bronchoscope, for example with the use of a coupler or adapter 202. The coupler or adapter 202 can be any suitable adapter for coupling the deployment or introducing device to the bronchoscope, such as a luer lock adapter. The introducing or deployment device 200 can have a handle assembly 204 that can have a number of different components for deploying an apparatus. For example, various embodiments of the handle assembly 204 can include an elongated tube with a lumen 206 for holding an apparatus such as a stent 207. The lumen 206 can have a deployment end region 208 and a control end region 210. The deployment end region 208 can house a stent 207 for deployment. In FIGS. 2A-2B, the stent 207 is shown in a deployed configuration. Before the stent 207 is deployed, however, the deployment end region 207 and the tube 206 may be inserted into the working channel 108 of the bronchoscope 100. The deployment end region 207 and at least some of the tube 206 may be advanced through the working channel 207 and the tube or insertion cord 106 of the bronchoscope 100. The coupler or adapter 202 may the couple to the coupling feature 112 of the adapter 110 and/or the fittings 109a, 109b, 109c (described in greater detail below).The control end region 210 can be positioned within the handle assembly and connected to a deployment release 210 that can be manipulated by the user to deploy the stent from the deployment end region 208.

The handle assembly 204 can also have a slide assembly 214 that can be configured to move or allow for adjustments to the placement of the stent 207. For example, the slide assembly 214 can allow for the movement of the handle assembly 204 with respect to the bronchoscope when it is attached. The slide assembly 214 can have a piston 218 that is fixed to the bronchoscope via the coupling features (202 and 112). The slide assembly 214 can also have a slide handle 220 that can move along the fixed piston 218 to adjust the position of the slide assembly 214, and handle assembly 204, with respect to the bronchoscope 100. Accordingly, the movement of the slide assembly 214 along the piston can adjust the position of the deployment end region 208. As illustrated in FIG. 2B, the handle assembly 204 can be positioned proximally with respect to the coupling feature 202 along the length of the piston.

In some embodiments, the slide assembly 214 can have a setscrew 222 that can be adjusted to fix the location of the slide assembly 214 and handle assembly 204. This can allow for adjustments in the position of the deployment end region 208 of the device 200 and likewise can result in the precise placement of the stent 207 or apparatus. Additionally, the set screw 222 can fix the position of the slide assembly 214 along the piston 218 and thus fix the position of the deployment end region 208 of the device 200. This fixing the position of the slide assembly 214 with the set screw 222 can avoid improper placement and the need to extended procedures and possible removal of the apparatus.

Additionally, some embodiments of the insertion device 200 can have one or more safety mechanisms that can be configured to engage and disengage with various features of the handle assembly and slide assembly to prevent unwanted movement or allow movement of the handle assembly 204 along the piston 218. For example, FIG. 2A illustrates two distinct removable safety tabs 224 and 226 that can be positioned at different longitudinal locations along a portion of the handle assembly. Each of the safety tabs can allow or prevent particular movement of the lumen 206 and the deployment end region 208 of the device 200 that can allow for proper deployment of the apparatus or stent 207. For example, with the first safety tab 224 in place the deployment end region 208 is unable to move with the depression of the release 210. Once the first tab is removed the depression of the release 210 can allow for partial movement of the deployment end region 208 that can result in a partial deployment of the apparatus or stent 207 until the second tab 226 is removed. The safety tabs can help to ensure the proper placement of the apparatus and prevent unwanted blockage of other channels.

FIG. 2B illustrates an alternative configuration of the handle assembly 204 having a release handle 211 with a free end 228 and a fixed end 230. The handle 211 can be rotatably connected to the handle assembly 204 such that the movement of the handle 211 can cause the movement of the deployment end region 208 and thus the deployment of the apparatus. Additionally, the safety mechanisms can have any number of configurations. For example, some embodiments may have buttons 232 and 234 that serve as a safety mechanism to prevent or allow partial movement of the deployment end region 208. Similar to the function of the safety tabs (224 and 226) the first and second safety buttons (232 and 234) can be depressed or activated to allow for the movement of the deployment end region 208 with the movement of the handle 211.

As can be appreciated, the slide assembly can be configured for one handed operation so that it can be easily moved or adjusted along the length of the piston by the practitioner or user to allow for more flexibility during operation. Accordingly, the slide assembly 214 and handle assembly 204 can have any number of configurations that can allow for ease and comfort of use by the user such that the device can be easily manipulated.

In the embodiment of FIG. 1A, the bronchoscope 100 is shown with an adapter 110 coupled to the bronchoscope 100 adjacent the proximal end region of the working channel 108. The adapter 110 may comprise coupling features 112 (e.g., threads) configured to couple to coupling features on a deployment device, such as coupling feature(s) 202 on the deployment device 200 of FIGS. 2A and 2B. One or both coupling features 112, 202 may comprise threads, including luer style threads. Snaps, detents, pins, collars, and other coupling features are likewise within the scope of this disclosure. Further, embodiments wherein a bronchoscope comprises a coupling feature (such as 112) integrally formed with the bronchoscope or fixedly attached thereto are likewise within the scope of this disclosure. Adapters 110 comprising a valve to prevent unwanted airflow or leakage through the working channel during use are likewise within the scope of this disclosure.

FIG. 1B illustrates a portion of the bronchoscope 100 with the adapter 110 removed and a first fitting 109a visible. The first fitting 109a may be fixedly secured to the protrusion on the bronchoscope 100 and may include or define a portion of the working channel 108 of the bronchoscope 100. The first fitting 109a includes a coupling feature configured to couple to the adapter 110. The coupling feature may include a neck extending from the bronchoscope 100 and a flanged head spaced from the bronchoscope. The neck may be substantially cylindrical with opposing flat sections. This configuration may allow an operator to selectively secure and/or remove the adapter 110 to the first fitting 109a.

FIG. 1C illustrates a second fitting 109b replacing the first fitting 109a on the bronchoscope 100 of FIG. 1B, according to an embodiment. FIG. 1D illustrates an embodiment of a deployment device 200′ coupled or secured directly to the second fitting 109b. The second fitting 109b may be fixedly secured to the protrusion on the bronchoscope 100 and may include or define a portion of the working channel 108 of the bronchoscope 100. The second fitting 109b may be configured to physically attach to one or more embodiments of the deployment device 200′ with no separate adapter or components. In some embodiments, the second fitting 109b includes a coupling feature configured to couple to the coupling feature on the deployment device 200′. The second fitting 109b may include an incomplete luer-type fitting. This luer-type fitting of the second fitting 109b may allow the handle of the deployment device 200′ to attach directly onto the bronchoscope 100, as shown in FIG. 1D. In this embodiment of the deployment device 200′ and the second fitting 109b on the bronchoscope 100, the physical attachment to the bronchoscope deployment device 200′ to the second fitting 109b of the bronchoscope 100 eliminates the need for separate adapters or components to couple the bronchoscope 100 to the deployment device 200′ (e.g., separate adapters or components are absent between the fitting 109b and the deployment device 200′).

FIG. 1E illustrates a cross-sectional view of an embodiment of a third fitting 109c configured to secure to a coupling region of a deployment device 200″, according to an embodiment. Though not shown in FIG. 1E, the third fitting 109c may be secured or coupled to the bronchoscope 100 similar to the first fitting 109a and the second fitting 109b. The coupling region illustrated in FIG. 1E replaces the coupling feature 202 on the deployment device 200 illustrated in FIGS. 2A-2B or may be configured to secure to the coupling feature 202 on the deployment device 200 illustrated in FIGS. 2A-2B. In some embodiments, the coupling region illustrated in FIG. 1E is formed on a distal end of the piston 218 and/or handle 220 of the deployment device 200.

In some embodiments, the fitting 109c is secured directly the bronchoscope 100. In other words, a secondary adapter is unnecessary to secure the deployment device 200″ to the third fitting 109c. In some embodiments, the third fitting 109c may include a scope luer secured to the bronchoscope 100. Accordingly, the third fitting 109c may include a neck and a flanged head extending from a protrusion on the bronchoscope 100. In some embodiments, the third fitting 109c is similar to or the same as the first fitting 109a shown in FIG. 1B.

One of the third fitting or the coupling region of the deployment device 200″ illustrated in FIG. 1E may include a tube 198 (such as a stainless steel component tube) inserted at least partially into the working channel 108 of the bronchoscope 100. This stainless steel tube may add stability to the handle of the bronchoscope 100. The coupling region of the deployment device 200″ illustrated in FIG. 1E may include fingers or levers 192 configured to selectively engage with the third fitting 109c to secure the deployment device 200″ to the third fitting 109c and the bronchoscope 100. For example, the levers 192 may include a first end region biased inward to engage with a flanged head 194 on the third fitting 109c. To release the deployment device 200″ from the third fitting 109c, a second end region of the levers 192 opposite to the first end region of the levers 192 may be pressed to disengage or release the first end region from the flanged head 194 on the third fitting 109c. This configuration of the coupling region of the deployment device 200″ allows for simple and quick attachment and release of the coupling region to the third fitting 109c. While shown with the third fitting 109c, the coupling region of the deployment device 200″ may be configured to detachably couple directly to any fitting (e.g., fitting 109a, 109b) described herein.

In any of the embodiments described above, during certain procedures, the deployment device 200 (or deployment devices 200′, 200″) can be coupled to the bronchoscope 100 such that it is fixed and relatively stationary with respect to the bronchoscope 100. In other words, the deployment device 200 can be supported by the body of the bronchoscope 100 such that movement of the deployment device 200 is limited. This, in turn may reduce unwanted distal stent movement while improving the practitioner's ability to maintain optimal scope and stent positioning during deployment. Reducing displacement of the deployment device 200 with respect to the bronchoscope 100 can aid in accurate placement of an airway stent. In some embodiments, the deployment device 200 and/or adapter 202 may be configured such that, when coupled to the bronchoscope 100, the deployment end region 208 of the deployment device 200 is just beyond the distal end region of the working channel 108. Additionally, or alternatively, the deployment device 200 may be couplable to the bronchoscope 100 along an adjustable range.

For example, in some procedures, a practitioner may first position the bronchoscope 100 adjacent a treatment area. The deployment device 200 can then be advanced through the working channel 108 and advanced such that the stent 207 is at the treatment location. The practitioner can confirm proper alignment of the deployment device 200, in particular the deployment end region 208 of the deployment device 200, via the optical components or system of the bronchoscope 100. The practitioner may then couple the deployment device 200 to the bronchoscope 100, fixing the position of the proximal end region of the deployment device 200 with respect to the distal end of the working channel 108 of the bronchoscope 108. For example, the coupler or adapter 202 may be secured to the coupler or adapter 110 to fix the position of the proximal end region of the deployment device 200 with respect to the distal end of the working channel 108 of the bronchoscope 100. In some embodiments, the coupler or adapter 202 may be configured to secure directly to the fittings 109a, 109b, or 109c. The practitioner can then maintain the position of the deployment device 200 and the deployment end region 208 (and thus the location at which the stent 207 will be deployed) by maintaining the position of the bronchoscope 100, as the deployment device 200 is fixed to the bronchoscope 100. Additionally, imprecise placement of the stent 207 due to movement between the deployment device 200 and bronchoscope 100 may be avoided.

Alternatively or additionally, in some therapies the deployment device 200 may first be coupled to the bronchoscope 100 with the stent 207 and/or the deployment end region 208 inside of the working channel 108 of the bronchoscope 100. As described above with respect to FIGS. 2A and 2B, the practitioner can advance the stent 207 and/or the deployment end region 208 to the delivery target after navigating to the delivery location by adjusting the length of the coupled portion of the deployment device 200 and fixing the length with the set screw 222. For example, the slide assembly 214 of the deployment device 200 adjacent the coupling portion 202 may comprise a column or piston that allows adjustment of the coupling portion 202 with respect to the stent pod and/or handle. That is, adjustment of the column or piston allows the deployment device 200 to be coupled to the bronchoscope 100, while still allowing a practitioner to adjust the relative position of the stent pod in the deployment end region 208 along a range. For example, to extend the stent pod in the deployment end region 208 from the bronchoscope 100 to position the stent pod at the desired treatment location. Embodiments wherein the relative position of the deployment device 200 and the bronchoscope 100 can be adjusted over a range (while the delivery device 200 is coupled to the bronchoscope 100) at any time during a procedure and locked at any point along the range are within the scope of this disclosure.

Further, coupling the deployment device 200 to the bronchoscope 100 may allow a single practitioner to view the portion of the deployment device 200 which contains the stent 207 (e.g., the stent pod) through the bronchoscope 100, controlling the bronchoscope 100 with one hand, and actuating the deployment device 200 with the other hand. (As compared to treatments where one user operates the bronchoscope 100 and a second user secures and adjusts the position of the deployment device 200 and actuates the deployment device 200 when signaled by the first user.) This can help prevent the stent 207 from being improperly placed within the lungs of the user. For example, if one person is viewing the location of the distal end of the deployment device in relation to the treatment area and another person deploying the stent, there may be error due to communication or due to a delay between the time when the first practitioner instructs the second practitioner to deploy the stent. Coupling the deployment device 200 to the bronchoscope 100 may enable one person to simultaneously control and confirm accurate scope position and deployment of the stent 207. Proper placement may be particularly relevant for covered or continuous stents that may close off side branches of the respiratory system if improperly placed, as further detailed below.

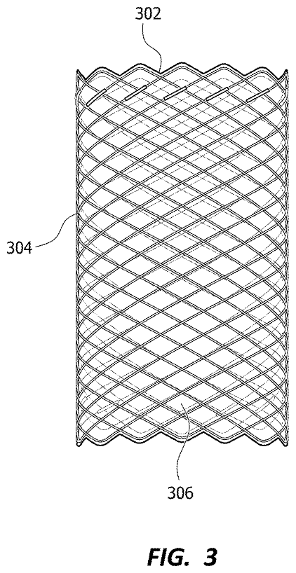

FIG. 3 illustrates an embodiment of a stent 302 that can be used in conjunction with the deployment device 200 illustrated in FIGS. 2A and/or 2B (e.g., the stent 207 may comprise the stent 302). The stent 302 can comprise a braided metallic scaffold portion 304. The braided scaffold portion 304 can allow the stent 302 to be compressed into a small form factor that can allow it to be deployed into small spaces such as the lungs. The deployment device 200 may comprise an outer sheath that is longitudinally displaceable with respect to one or more inner sheathes. The stent 302 can be compressed into a position between an inner sheath and the outer sheath (the “stent pod”) of the deployment device 200 such that the stent 302 is coupled to and contained within the deployment device 200. The stent 302 may be self-expanding such that upon retraction of the outer sheath with respect to the inner sheath and the stent, the stent 302 expands and engages the body structure surrounding it. FIGS. 2A and 2B illustrate a stent 207 partially deployed from the deployment device 200. That is, in the position of FIGS. 2A and 2B, the outer sheath is partially retracted and the stent 207 partially expanded or deployed. The stent 302 may deploy similar to the deployment of the stent 207 shown in FIGS. 2A and 2B. When expanded within an airway, the stent 302 may be used to treat portions of the airway, for example by opening airway obstructions and maintaining flow between airway branches. In various embodiments, the stent 302 may comprise a covering 306 disposed on the scaffold portion 304. This cover 306 can allow the stent 302 to be deployed within the body and not cause irritation to the tissues of the lungs as well as limiting ingrowth of tissue through the scaffold 304. The covering 306 can be hydrophobic or hydrophilic, including embodiments wherein a base covering material is treated with a hydrophobic or hydrophilic coating adaptable for use with human tissues. Coatings may also be anti-microbial or used to dispense anti-inflammatory drugs or biological therapies.

Sizes and configurations of the stent 302 can take on any form that can be suitable for use within the lungs. For example, in some embodiments the scaffolding portion 304 can be made from a nitinol material that can be preformed into any desired shape. While the shape illustrated in FIG. 3 is cylindrical, it can be appreciated that nitinol can be formed into any shape and heat set in order to return to that shape. The nitinol can then be compressed into a small form factor and upon activation will return to the heat set shape. Other embodiments of the scaffolding portion 304 can be made from platinol, any suitable metallic material, polymeric materials, and so forth. Additionally, various embodiments can have a braided configuration or any other suitable configuration that can be deployed. For example, the braided configuration can be made from many different wires that are braided around a mold to form the desired shape. The number of wires can vary as can the diameter of the wires. In accordance with some embodiments, the stent 302 can have any suitable diameter for placement in an airway.

The outer coating or covering 306 of the stent 302 can also be from any suitable material and or configuration. Many embodiments of the covering 306 can be made from a silicone type material. Some embodiments can also include different structures and/or markers that can help position the stent 302 within an airway. For example, some embodiments can have a texturing formed from microstructures on the exterior surface of the covering 306, including microprinting on the outside surface of the covering 306. This texturing can help the stent 302 couple to the lining of the airway and hold the stent 302 in position during placement and after deployment. This holding of the stent 302 in position during placement and after deployment can be beneficial because it can help prevent the stent 302 from moving if external factors attempt to move the stent 302. Additionally, the covering 306 may be configured to facilitate mucociliary clearance. For example, the covering 306 may comprise patterns, including micro-printed patterns on an inside surface of the covering that facilitate mucociliary clearance.

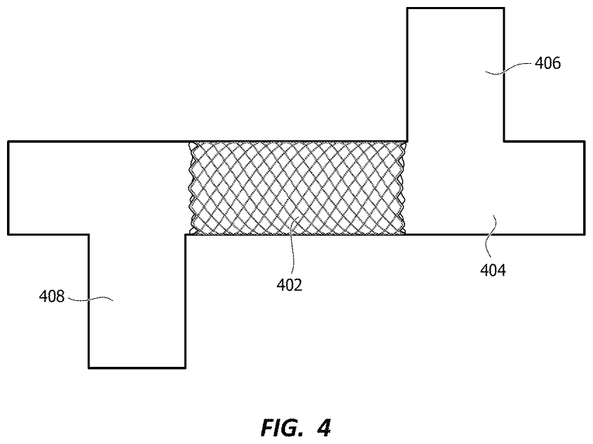

As has been discussed, the accurate deployment of the stent 302 may enable a practitioner to treat a lesion adjacent a branch in the airway without cutting off flow to the airway. For examples, in stents designed to open an airway, the branched nature of the lungs can pose potential issued when deploying stents. If the stent is not properly placed it can cause partial or complete blockages of adjacent airways. FIG. 4 schematically illustrates a placement of a stent 402 within an airway 404 of a patient. Unless otherwise noted, the stent 402 may include any aspect of the stents 207, 302. The airway 404 can have one or more adjacent airway passages 406 and 408 that branch off of the airway 404. FIG. 4 is a simplified version of a branching system that can be found in the lungs. As can be appreciated, the placement of the stent 402 can be important so as not to block the adjacent airway passages 406 and 408 if the desired location is associated with such passages. Accordingly, having a deployment device, similar to the deployment device 200 illustrated in FIGS. 2A and 2B coupled to the bronchoscope, can be helpful in ensuring the precise placement of the stent 402 within the airway. Precise placement, in turn, may help to facilitate treatment to locations adjacent branches passages (408 and 406) without cutting off fluid communication between airway 404 and branches 408 and/or 406.

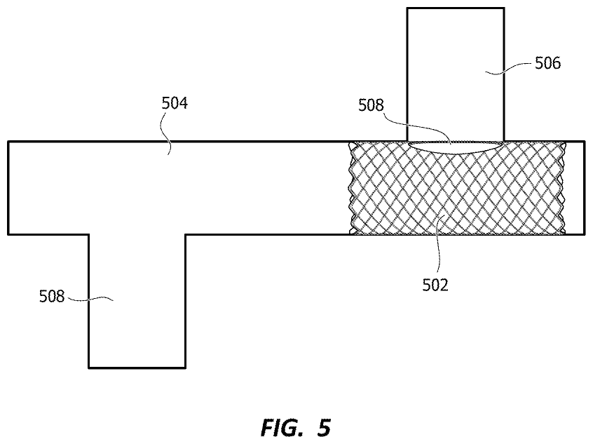

In some embodiments, a stent has one or more openings or selectively openable sections configured for placement in a branched airway. For example, FIG. 5 illustrates placement of a stent 502 within an airway 504 where the stent 502 is positioned over an adjacent airway passage 506. Unless otherwise noted, the stent 502 may include any aspect of the stents 207, 302, 402. The stent 502 may be configured for use when a lesion or some feature of the anatomy necessitates placement of the stent 502 such that a body of the stent 502 would cover a branched airway. In other words, the placement of the stent 502 may need to be across an adjacent airway passage 506 in order to provide the desired relief from the damaged or problematic area. As such, some embodiments of a stent 502 can have an opening or aperture 508 in a sidewall of the stent 502. The opening or aperture 508 can be in a covering (such as cover 306 of FIG. 3) or can also extend through any supporting structure of the stent 502. In some embodiments, the opening or aperture 508 may be sized equivalent or smaller than gaps between the wires of a scaffolding (such as 304) of a stent. The opening or aperture 508 can allow for the flow of air to take place within the main channel of the stent 502 from the adjacent airway passage 506 such that the placement of the stent 502 does not interfere with the overall gas exchange of the patient. This configuration can also be helpful if and when a stent 502 is placed where the precision placement may have been disrupted or if the stent is moved over time.

In some instances, stents (such as stent 502) with predefined openings or apertures can require more precise deployment mechanisms in order to allow a doctor to properly deploy the stent without such that the opening 508 is properly positioned. Some embodiments of a deployment device, similar to the deployment device 200 illustrated in FIGS. 2A and 2B can have an indicator mechanism that is positioned on the handle or some other area of the deployment device that corresponds to the location of the opening or aperture 508. In other embodiments, the position indicator can be on the stent 502 itself. For example, some embodiments may have an indicator on one or both ends of the stent 502 that can be seen through the optical components of the bronchoscope such that the user can identify the position of the opening or aperture 508 within the airway 504. The indicator can be any suitable indicator such as a physical indicator or some can be an illuminated indicator that can be illuminated by a light. The illuminated indicator can be a visible marker, a radiopaque marker, or so forth and can be placed at one end or both of the stent. In some embodiments, the indicator indicates that the user may need to rotate the catheter (including the lumen 206) while attached to the scope such that proper alignment is maintained at the stent pod in the deployment end region 207 to lung opening.

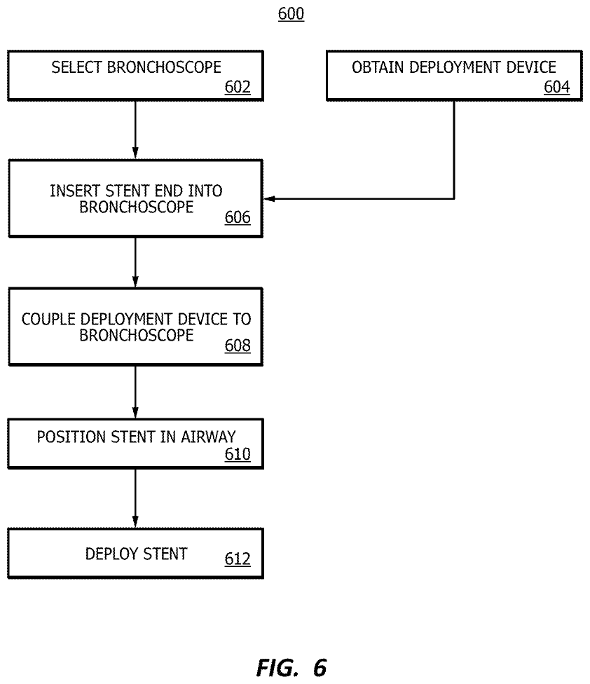

Referring now to FIG. 6 through FIG. 8, flow diagrams of embodiments of methods of use are provide. The embodiments of methods of FIGS. 6-8 may include any bronchoscope, deployment device, stents and/or couplers or adapters disclosed herein.

FIG. 6 for example illustrates a process 600 of deploying a stent in accordance with some embodiments. The user can obtain 602 a bronchoscope as well as obtain 604 a deployment device. A stent end region of the deployment device can be inserted 606 into a working channel of the bronchoscope, and the deployment device can then be coupled 608 or attached to the bronchoscope. The stent deployment end region of the deployment device can then be moved through the working channel and tube of the bronchoscope, and positioned 610 into the airway of a patient by changing the length of the adjustable coupling of the deployment device to the bronchoscope. With the deployment device coupled to the bronchoscope the doctor or user can operate the bronchoscope to the desired location of the stent in the deployment end region of the deployment device. This operation of the bronchoscope to the desired location can be done without concern that the deployment device will be accidentally adjusted or moved. Once the deployment end region of the deployment device is positioned 610, the stent in the deployment end region can be deployed 612 to the precise location as desired.

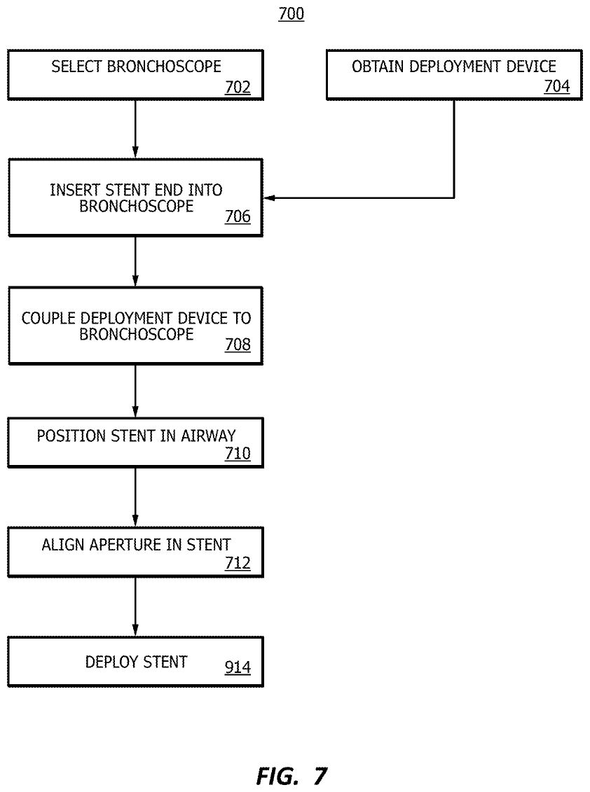

FIG. 7 illustrates a modified deployment process 700 from the process 600 as illustrated in FIG. 6. For example, the user can obtain 702 a bronchoscope as well as obtain 704 a deployment device. The stent end of the deployment device may be inserted 706 into the working channel of the bronchoscope, and the deployment device can be coupled 708 to the bronchoscope. The stent, in some embodiments can have an aperture that can be configured to be positioned at a location where two adjacent airways are present. The stent can be positioned 710 within the airway and the aperture can be aligned 712 with one of the adjacent airways to allow for the flow of air to continue with the stent present. Once the stent is in position it can be deployed 714.

In some embodiments, the deployment device (e.g., the deployment device 200 of FIGS. 2A and 2B) may comprise one or more indicia on the handle or a proximal portion of the deployment device to indicate whether the stent pod has fully exited the working channel of the bronchoscope. In other words, indicia configured to prevent or minimize the premature deployment of the stent within the working channel can be printed or otherwise disposed on a proximal portion of the deployment device. Additionally or alternatively, fluoroscopic or other indicia configured for the same purpose may be disposed on other portions of the deployment device. Such indicia may be used when coupling the deployment device to the bronchoscope to ensure or confirm coupling at the right location. Additionally or alternatively, such indicia may allow a practitioner the option to deploy a stent without coupling the deployment device to the bronchoscope if the event of an unexpected need to modify the deployment procedure.

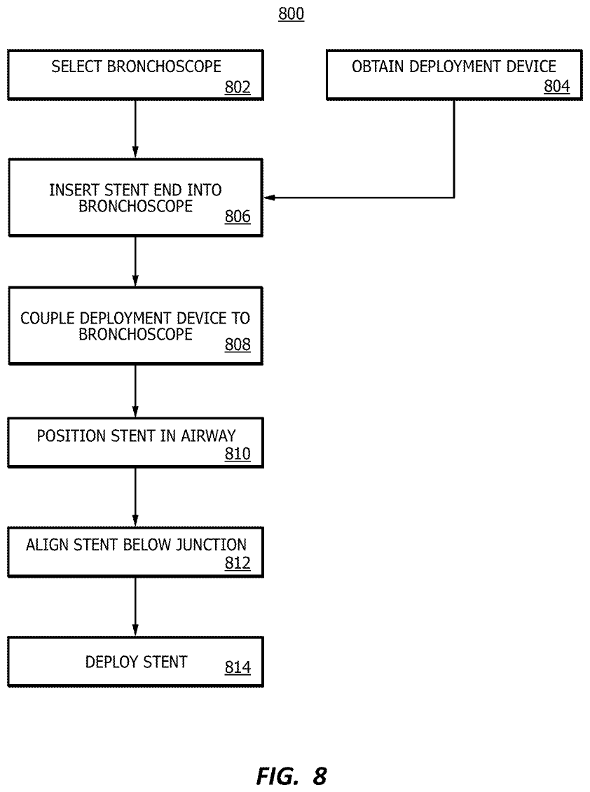

Similarly, FIG. 8 illustrates a process 800 by which a stent can be positioned in between multiple airways. In some embodiments of the process 800, the user can obtain 802 a bronchoscope as well as obtain 804 a deployment device. The stent end of the deployment device may be inserted 806 into the working channel of the bronchoscope, and the deployment device can be coupled 808 to the bronchoscope. The relative position of the deployment device to the bronchoscope can be adjusted to position 810 the stent within the airway of the user. In some embodiments, the stent can be aligned 812 below a junction of adjacent channels such that the stent is positioned at a precise location that is between various adjacent channels and does not block either of the airway channels. Once in the desired location, the stent can then be deployed 814.

The coupling of the deployment device, as illustrated, throughout can be advantageous in the positioning of an airway stent. This can allow a user to precisely position a stent within the smaller branched airway channels of the patient. Additionally, the adjustable coupling column can allow the user to make minute adjustments to the position and location of the stent such that a stent with an opening can be placed between adjacent airway channels or passages. This can allow a doctor to use different sizes of stents and still achieve the desired outcome for the patient.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents

What is claimed is:

1. A method of treating an airway, the method comprising:
obtaining a deployment device, wherein the deployment device contains an airway stent positioned at a first end, the deployment device having a second end with a release handle;
introducing the first end of the deployment device into a working channel of a bronchoscope;
removably coupling the deployment device to the working channel of the bronchoscope;
introducing the first end of the deployment device into an airway passage of a patient by adjusting the position of the deployment device with respect to the bronchoscope while the deployment device is coupled to the bronchoscope;
actuating the release handle of the deployment device to release the airway stent into the airway passage of the patient, such that the airway stent is positioned to hold open the airway passage of the patient.

2. The method of claim 1, wherein the release handle comprises an indicator that correlates to an orientation of the airway stent.

3. The method of claim 1, wherein the airway stent is a braided covered airway stent.

4. The method of claim 3, wherein the braided covered airway stent has a metallic scaffold portion covered by a flexible covering, the metallic scaffold portion comprising nitinol.

5. The method of claim 3, wherein the braided covered airway stent further comprises an aperture in a sidewall portion providing fluid communication between an internal channel of the airway stent and an exterior surface of the braided covered airway stent.

6. The method of claim 5, wherein the aperture is disposed at an opening of the braided metallic scaffold of the braided covered stent.

7. The method of claim 5, further comprising inserting the braided covered airway stent precisely at a branch location between at least two airway passages within the patient such that the aperture is positioned adjacent at least one of the two airway passages.

8. The method of 5, wherein the release handle comprises an indicator that correlates to an orientation of the aperture.

9. The method of claim 1, further comprising inserting the airway stent precisely beyond a branch between two airway passages within the patient.

10. The method of claim 1, wherein removably coupling the deployment device to the bronchoscope comprises coupling an adapter interface to the bronchoscope such that the adapter is removably connected to the bronchoscope.

11. The method of claim 1, wherein removably coupling the deployment device to the bronchoscope comprises coupling the deployment device directly to an integral component of the bronchoscope.

12. The method of claim 1, wherein the airway stent comprises a covering including microprinting on an inside surface of the covering.

13. The method of claim 1, wherein the airway stent comprises a covering including microprinting on an outside surface of the covering.

14. A method for treating an airway blockage comprising:
providing a bronchoscope comprising a working channel;
coupling an adapter to the working channel;
coupling a stent deployment device to the adapter, wherein the stent deployment device contains an airway stent positioned at a first end, the stent deployment device having a second end with a release handle; and
deploying the airway stent into the airway of the patient.

15. The method of claim 14, wherein the airway stent is a braided airway stent having a metallic scaffolding portion including nitinol.

16. The method of claim 14, wherein the airway stent is a braided covered airway stent.

17. The method of claim 16, wherein the braided covered airway stent is covered with a hydrophilic coating.

18. The method of claim 16, wherein the braided covered airway stent comprises a covering including microprinting on an inside surface of the covering.

19. The method of claim 16, wherein the braided covered airway stent comprises a covering including microprinting on an inside surface of the covering.

20. The method of claim 16, wherein:
the airway stent comprises an aperture disposed in an exterior surface of the braided covered airway stent; and
the method further comprises aligning an indicia on the deployment device such that an aperture on the airway stent is disposed in a desired orientation.
